(19) United States
(12) Patent Application Publication
(10) Pub. No.: US 2008/0192202 A1
(43) Pub. Date: Aug. 14, 2008

(54) Device and Method for Investigating Changes in the Eye

(76) Inventor: Christopher Paul Lewkowski, Western Australia (AU)

Correspondence Address: PAUL, HASTINGS, JANOFSKY & WALKER LLP, 875 15th Street, NW, Washington, DC, 20005, US

(21) Appl. No.: 11/666,452

(22) PCT Filed: Oct. 31, 2005
(86) PCT No.: PCT/AU2005/001671

§ 371 Date: Nov. 13, 2007

(30) Foreign Application Priority Data
Nov. 1, 2004 (AU) 2004906285

(51) Int. Cl. A61B 3/113

(52) U.S. Cl. 351/206; 351/246

(57) ABSTRACT

A mask (10) has a lightweight, robust, plastics or metal frame or body (12) on which other components are supported. The body (12) includes straps or arms (14) to allow the mask to be temporarily attached to the user's head and is designed to provide a light omitting cover for the user's eyes. A suitable light source such as an infrared light source (20) is provided to illuminate the user's eyes (22). A pair of lenses (30) is provided to focus images of the user's eyes onto a pair of image sensors (40). There is also described a method of diagnosing various disorders by investigating changes in the eye using the mask (10). The portable device (10) can be used for the investigation of nystagmus or more generally in oculography, and for the investigation of other changes in the eye.

### FIELD OF THE INVENTION

The present invention relates to the investigation of changes in the eye and relates particularly, though not exclusively, to a device for the investigation of nystagmus which may be associated with vestibular and other neurological disorders, and a method of conducting the investigation using the device.

### BACKGROUND TO THE INVENTION

The presence of a number of medical and health problems can be detected by changes in the eye, including unusual changes in the position, movement and dilation of the eye. For example, it has been found that one of the leading causes of dizziness and balance problems is associated with the vestibular system in the inner ear. The presence of nystagmus (very specific, rapid, involuntary eye movements) during a dizziness attack can suggest to a clinician that there is a vertiginous component to it and the direction of the nystagmus may provide some evidence to a specialist in the field, of more specific information, such as which ear (or which part of the ear) has the active disease.

Dizziness and balance problems constitute a major public health problem. A significant proportion of adults have had an episode of dizziness that occurs with enough intensity or frequency to promote a visit to the doctor. It is one of the most difficult complaints to assess, as it is a subjective symptom of potentially numerous causes. Dizziness is often an episodic symptom, with the frequency of episodes highly variable. As a patient will very rarely have an episode in the clinic, clinicians are frequently forced to rely solely on the patient's (often unintentionally misleading) report of the symptom.

Accordingly, accurate diagnosis of balance symptoms is important not only to exclude potentially serious central causes but to aid successful treatment. Unfortunately diagnosis is sometimes not possible, or delayed. Studies have shown that general practitioners (GPs) rarely failed to refer urgent cases but often failed to refer patients with persistent vestibular conditions. Part of the difficulty is that prior art nystagmography apparatus for detecting and recording nystagmus are large, expensive machines that are only available for use by specialists. Due to the size, complexity and expense of these machines they are limited to laboratory use for specific tests. These machines are rarely used to monitor patients at the time of a dizzy attack.

The present invention was developed with a view to providing a portable device for the investigation of nystagmus and a method of conducting the investigation using the device. The device can be used away from the clinic and does not need a specialist to operate it. However it will be understood that the device may also be used more generally in oculography and for the investigation of other changes in the eye.

References to prior art in this specification are provided for illustrative purposes only and are not to be taken as an admission that such prior art is part of the common general knowledge in Australia or elsewhere.

### SUMMARY OF THE INVENTION

According to a first aspect of the present invention there is provided a self-contained portable mask to provide a light omitting cover for the eyes for use in the investigation of changes in the eye such as nystagmus, the mask further comprising:

a suitable light source to illuminate the wearer's eyes;

a lens arrangement to focus images of the wearer's eyes;

a digital image sensor to receive the focused images;

a data processing unit for processing the sensed images;

a storage medium to record the processed images; and,

a communications port for communicating images and commands to an external receiver.

The mask may also comprise an inbuilt user interface for giving commands or instructions to the data processing unit. The mask may also comprise a status indicator to communicate its status and/or the quality of the recorded images to the user. Preferably the storage medium is removable from an inbuilt port.

The mask may enjoy the following advantages: It is lightweight, durable and easy to hold and use. It is able to record and store images from both eyes simultaneously for a suitable length of time. The images captured will be clear and have a resolution suitable for analysis. The images will be compatible with existing analysis techniques. The mask will also be inexpensive to manufacture.

The mask can be configured to store the images on the internal storage medium. Alternatively, or in addition, the images may be sent in real time over the communications port to a personal computer (PC) or other receiving device for analysis or viewing.

The communication port may be wired (including but not limited to Ethernet, USB, IEEE 1934) or wireless (including but not limited to Bluetooth, Wireless LAN).

Use of the mask also opens telemedicine opportunities, since the images can easily be sent via the Internet to a specialised clinician who will interpret the results. It may also provide an excellent tool for teaching and record keeping purposes.

According to a further aspect of the present invention there is provided a method of diagnosing various disorders by investigating changes in the eye using the mask, the method comprising the steps of:

supplying the mask to a wearer who may be suffering from a particular disorder;

deploying the mask over the wearer's eyes when symptoms of the disorder may be present;

capturing digital images of the wearer's eyes for a period of time immediately after deploying the mask;

compressing the digital images; and,

communicating the compressed images from the mask to an external receiver for analysis.

Preferably the method comprises the further step of temporarily storing the compressed digital images in the mask.

According to a still further aspect of the present invention there is provided a method of investigating changes in the eye using the mask, the method comprising the steps of:

supplying the mask to a clinic;

administering the mask to a patient;

receiving compressed digital images of the eye from the mask; and,

analysing the images for the purpose of diagnosing a particular disorder or medical condition.

The mask may be returned to the supplying clinic for analysis of the recorded images. Alternatively, a storage medium holding the stored images may be removed from the mask and transported to the supplying clinic for analysis of the images.

The analysis may be aimed at diagnosing vestibular and neurological disorders. Typically the analysis will concentrate on recorded eye movements during an attack of dizziness. In particular the analysis may involve the identification of nystagmus during the time of a vestibular attack, as well as the intensity and direction of the nystagmus. Analysing eye movements during an attack of dizziness can be a key diagnostic tool in vestibular disorders. It may help determine whether the cause of a patient's imbalance problems is vestibular (inner ear) or otherwise, and if vestibular they may point towards a specific diagnosis.

The analysis of the captured images of changes in the eye may also help to diagnose other neurological disorders. Changes in other attributes of the eye such as discolouration, pupil size and iris size may also lead to diagnosis of numerous other medical conditions.

Throughout the specification, unless the context requires otherwise, the word “comprise” or variations such as “comprises” or “comprising”, will be understood to imply the inclusion of a stated integer or group of integers but not the exclusion of any other integer or group of integers. Likewise the word “preferably” or variations such as “preferred”, will be understood to imply that a stated integer or group of integers is desirable but not essential to the working of the invention.

### BRIEF DESCRIPTION OF THE DRAWINGS

The nature of the invention will be better understood from the following detailed description of preferred embodiments of the mask and method of using the same, given by way of example only, with reference to the accompanying drawings, in which:

FIG. 1 is a schematic diagram illustrating the basic principles of a mask in accordance with the present invention;

FIG. 2 illustrates a preferred embodiment of the mask in accordance with the present invention;

FIG. 3 is a functional block diagram of the internal electronic components in the mask of FIG. 2; and,

FIG. 4 is a flow chart illustrating a preferred method of using the mask of FIG. 2.

### DETAILED DESCRIPTION OF PREFERRED EMBODIMENTS

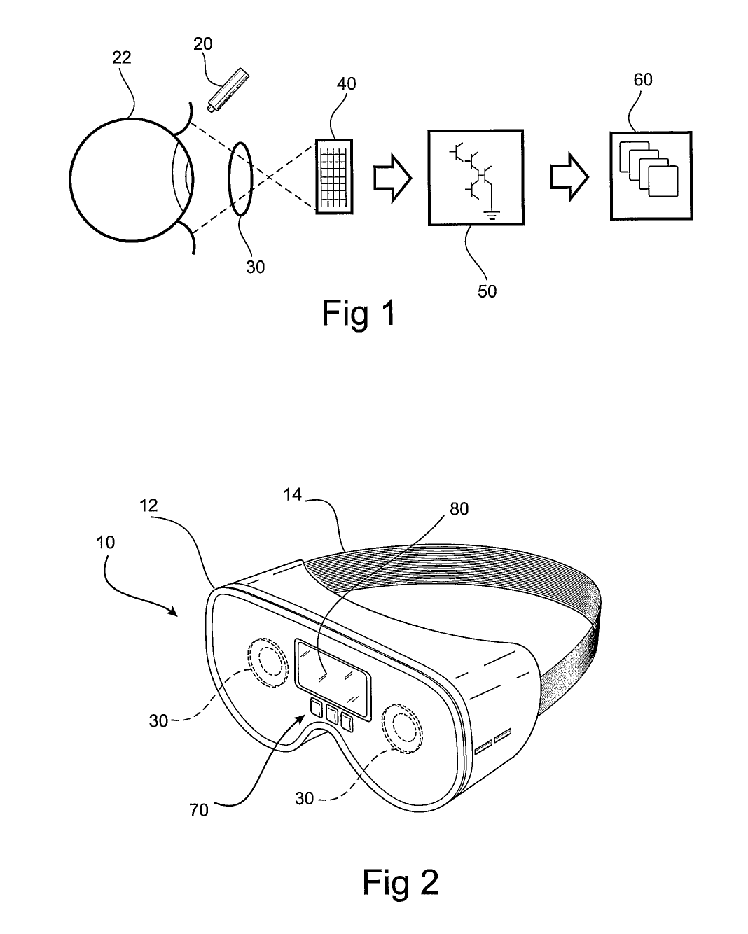

A preferred embodiment of a mask 10 according to the present invention, as shown in FIGS. 1 and 2, comprises a lightweight, robust, plastics or metal frame or body 12 on which the other components are supported. The body 12 preferably includes straps or arms 14 to allow the mask to be temporarily attached to the user's head. Preferably the mask 10 is designed to provide a light omitting, tightly fitting cover for the user's eyes. In order to detect nystagmus it is useful to block out all external light stimulation to the eyes, as the user could override the spontaneous nystagmus reflex by fixating on a point. A suitable light source 20 is provided to illuminate the user's eyes 22.

Preferably an infrared light source 20 is employed. The infrared light source 20 may be light emitting diodes (LEDs) mounted within the body 12, a set of one or many provided for each eye.

A pair of lenses 30 is provided to focus images of the user's eyes onto a pair of image sensors 40. The factors to be considered in choosing a suitable lens are focal length, sensor size, distance to image plane, image size and aperture (light). One particular preferable requirement of the lens is to have a depth of field that will allow the eye image to be always in focus. As different users will have varying length between the lens and eye, the lens will usefully have a depth of field over this range. Another possibility is for the lenses 30 to use auto-focusing technology or to allow the focus to be adjusted by the wearer and/or a clinician.

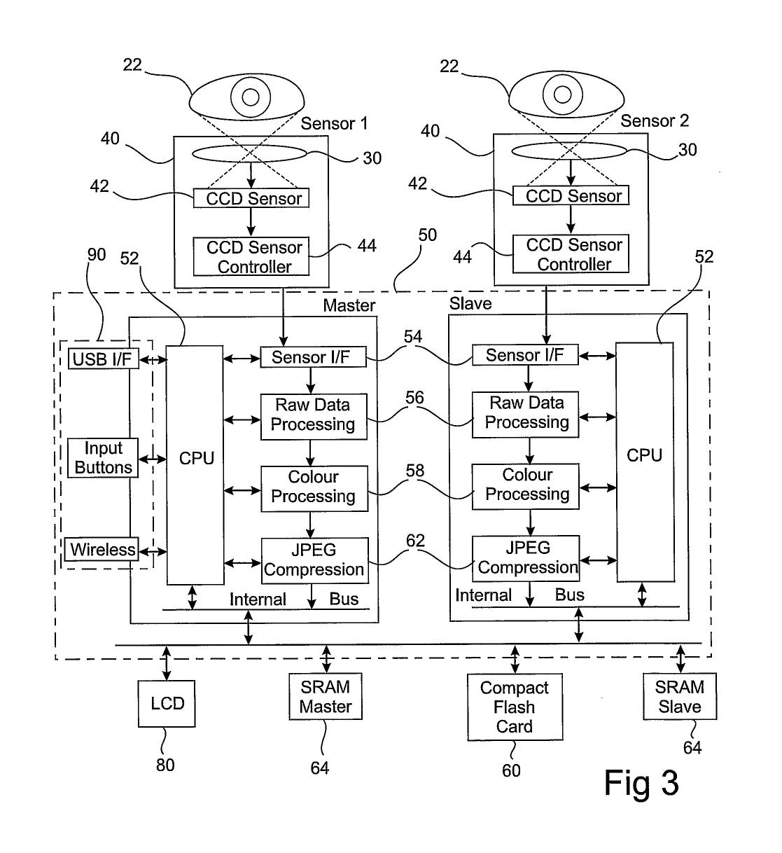

The image sensors in this embodiment are digital image sensors 40, and currently CMOS or CCD technologies are suitable. Each image sensor 40 typically includes a CCD sensor 42 and a CCD sensor controller 44 as shown in FIG. 3. Preferably the lenses 30 are incorporated into or attached to the image sensors 40. The image sensors 40 are operated to capture images at a predefined frame rate. The raw images captured by the image sensors 40 are transferred synchronously to a digital image processing unit (IPU) 50. The IPU 50 may correct the images received for such photographic problems as over-exposure or under-exposure, bad pixels, etc. It will then compress the captured images using an industry standard compression technology. Currently this is the JPEG standard, however it is to be understood that any suitable compression technology may be employed.

Operation of the IPU 50 will now be described in more detail with reference to FIG. 3. The IPU 50 is responsible for coordinating the collection, manipulation, compression, sequencing and transfer of the images of both the user's eyes 22. It will include various electronic components that will facilitate these functions. Preferably the IPU 50 employs dual CPUs 52 operated in a Master/Slave configuration for processing the left and right eye images respectively. Raw image data is received from the respective image sensors 40 via sensor I/F devices 54 and processed in raw data processing devices 56 and colour processing devices 58. The raw data processing devices 56 and colour processing devices 58 will adjust the images to compensate for light variations and colour matching. The processed images are then compressed using a compression device 62. The compression method may include JPEG, and any other suitable compression algorithms. Compression ratio is configurable based on clarity and speed requirements. The sequence of compressed images is then either stored within the devices internal memory, in this case static random access memory (SRAM) 64, or on a removable digital media device or transferred to an external receiver via a communications port 90.

Preferably, if a removable storage device is used, the JPEG compressed images are written to an industry standard portable storage media device 60. Currently a compact flash (CF) card 60 is used as the storage medium but other types of storage media could be used. The main reason for using this memory type is that it is currently widely available and has a large memory capacity. It is also relatively large in physical dimensions and therefore easy to handle. This is important as elderly and technologically challenged wearers may be changing the memory device by themselves, for instance, to replace it with a fresh one when it is full.

Preferably a user interface 70 is built into the mask for inputting commands or instructions to the IPU 50 (see FIG. 1). This could include a keypad, a series of input switches or dials, or a touch screen. The user interface 70 may also include a status indicator, which may consist of an LCD screen 80, LEDs, a speaker or other audio/sound device, which the IPU 50 can use to indicate its status and/or the quality of the recorded images to the user. The LCD screen 80 may permit the user, their spouse, or a clinician to easily ascertain whether or not the device is correctly recording the images of both eyes. In addition the user may view the stored images to confirm that the recording is correctly working, or a clinician may quickly review the images prior to downloading and transmission to a specialist laboratory for analysis.

There is also a wired and/or wireless communication port 90 built into the mask 10 for downloading images from the CF card 60 at a later time, or in real time as they are captured. Typical technologies for the communications port 90 include, but are not limited to, USB, IEEE 1934, Ethernet, Bluetooth or Wireless LAN.

Rechargeable or replaceable batteries are included (not visible) in the mask 10 to provide electrical power.

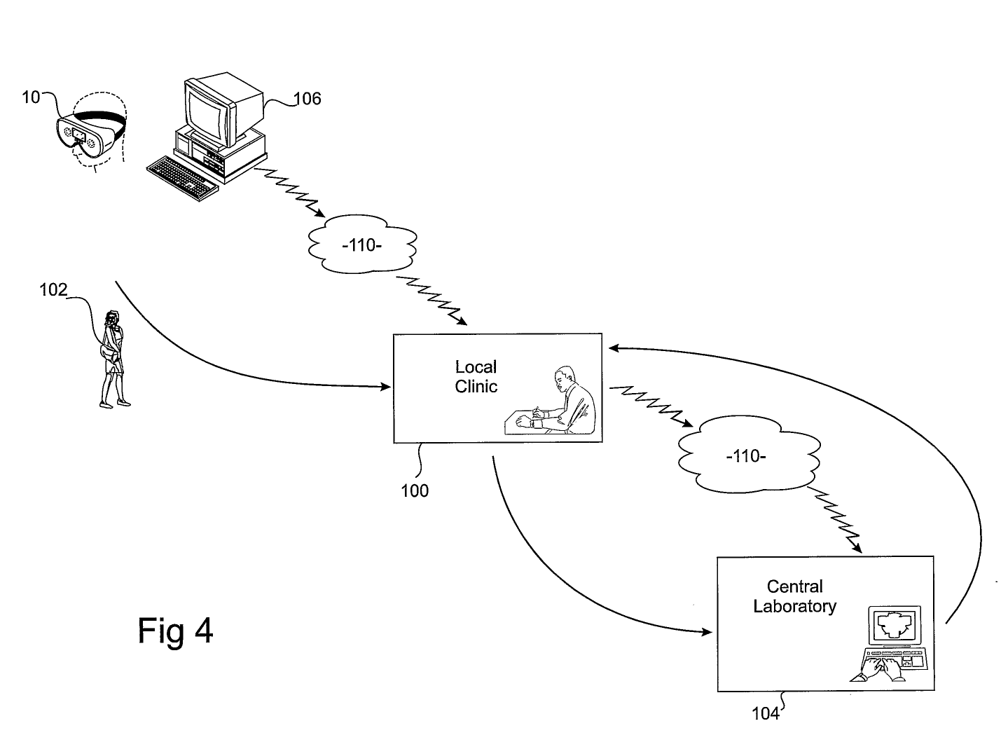

Preferred methods of investigating various neurological and other disorders using the mask 10 will now be described with reference to FIG. 4. In one preferred method of the invention, a clinic 100 will provide the mask 10 to a patient 102 under investigation for them to carry home or with them where ever they go. The clinic 100 will also be responsible for training the patient or carer to make themself safe and to deploy the mask when they experience an attack of dizziness commencing. Deploying the mask involves placing the mask over the head in front of the eyes. The mask 10 is then held in place by the user, or using the strap 14. Activating the mask involves pressing a button, or applying a command via the communications port, that both activates the light source 20 to illuminate the patient's eyes 22 and the image sensors 40. The image sensors 40 will capture images of the eyes for a predetermined period of time and will send these images to the processor 50 for processing and storage in the storage media 60.

The mask 10 may subsequently be returned to the clinic 100 by the patient 102 for analysis of the recorded images. The clinic 100 can download the images using the communications port 90 or remove the CF card 60 and download the images from that. Alternatively, if the patient lives in a remote location, they may be able to download the images onto their home or remote clinic's personal computer 106 and transmit the compressed images to the clinic 100 via the internet 110. The image analysis may be performed in the clinic 100 if it has the necessary specialised personnel. Alternatively, the images may be transmitted via the internet to a central laboratory 104 for analysis by specialists. In the case of nystagmography the analysis concentrates on recorded eye movements during the dizzy attack. The analysis may make use of already existing eye tracking processing software which is able to analyse the eye images for vertical, horizontal or tortional eye movements, pupil diameter and other eye attribute parameters.

Another mode of use for the device is the real-time viewing and recording of eye images. This would involve a clinician or patient holding or attaching the device 10 to the patient's head in a local clinic 100. After processing the images the device 10 will send the images via the communications port 90 to a PC in the clinic. The clinician can then choose to view, save, analyse and/or transmit these images to the central laboratory 104. The local clinic 100 may be a GP's clinic, a hospital emergency room, 24 hour medical clinic, remote clinic or specialist clinic, which has one or more of the devices 10 available in the clinic to perform a quick test.

In a more advanced version of the device 10, additional processing power is provided onboard the IPU 50 to perform programmed analysis of the stored images and to automatically generate a preliminary report that can be subsequently verified by a specialist if necessary. Alternatively, suitable analysis software may be supplied to the clinic or user for use in a stand alone processing unit (such as a docking station for the device), or in a desktop PC. The stored images can then be downloaded to the stand alone unit or PC for automatic analysis.

Although the preferred embodiment of the device and method of use have focused on the investigation of eye movement for vestibular and neurological disorders during the time of an episode, there are many other potential uses for the mask 10 including, but not limited to, the following:
(a) bedside monitoring of hospital in patients
(b) telemedicine applications for remote areas
(c) monitoring the effect of drugs on the reduction of symptoms
(d) wireless/hands free viewing of eye movements during postulography and manoeuvres such as Hallpike's manoeuvres
(e) teaching tool
(f) record keeping tool
(g) research tool

Now that preferred embodiments of the mask and method of use have been described in detail, it will be apparent that it provides a number of advantages over the prior art, including the following:

(i) The mask is relatively inexpensive to manufacture and therefore can be made available at an affordable price to a broader clientele

(ii) The mask is highly portable and therefore can be supplied to users to take home for self-administration

(iii) It can be used in conjunction with a conventional desktop or laptop personal computer and therefore reduces additional hardware costs

(iv) It is self-contained, with the video images being compressed on board so that no external storage media are required

(v) The mask is lightweight, durable and easy to hold and use.

It will be readily apparent to persons skilled in the relevant arts that various modifications and improvements may be made to the foregoing embodiments, in addition to those already described, without departing from the basic inventive concepts of the present invention. For example, this invention can also be used for monitoring changes in other attributes of the eye such as pupil size, eyelid movement, iris discolouration, etc. Therefore, it will be appreciated that the scope of the invention is not limited to the specific embodiments described and is to be determined from the appended claims.

What is claimed is:

1. A self-contained portable mask for use in the investigation of changes in the eye such as nystagmus, the mask comprising:
a light omitting cover for the eyes;
a light source to illuminate the wearer's eyes;
a lens arrangement to focus images of the wearer's eyes;
a digital image sensor to receive the focused images;
a data for processing the sensed images;
a storage medium to record the processed images; and,
a communications port for communicating images and commands to an external receiver.

2. A self-contained portable mask according to claim 1, further comprising an inbuilt user interface for giving commands or instructions to the data processing unit.

3. A self-contained portable mask according to claim 1, further comprising a status indicator to communicate its status and/or the quality of the recorded images to the user.

4. A self-contained portable mask according to claim 1, wherein the storage medium is removable from an inbuilt port.

5. A self-contained portable mask according to claim 1, wherein the data processing unit compresses the digital images prior to storing the processed images on the internal storage medium.

6. A self-contained portable mask according to claim 1, wherein the data processing unit is configured to send the images in real time over the communications port to a personal computer or other external device for analysis or viewing.

7. A method of diagnosing various disorders by investigating changes in the eye using the mask of claim 1, the method comprising the steps of:
supplying the mask to a wearer who may be suffering from a particular disorder;
deploying the mask over the wearer's eyes when symptoms of the disorder may be present;
capturing digital images of the wearer's eyes for a period of time deploying the mask;
compressing the digital images in the mask; and,
communicating the compressed images from the mask to an external receiver for analysis.

8. A method of diagnosing various disorders by investigating changes in the eye according claim 7, comprising the further step of temporarily storing the digital images in the mask.

9. A method of investigating changes in the eye using the mask of claim 1, the method comprising the steps of:
administering the mask to a patient;
receiving compressed digital images of the eye from the mask; and,
analysing the images for the purpose of diagnosing a particular disorder or medical condition.

10. A method of investigating changes in the eye according to claim 9, further comprising the step of transmitting the compressed digital images to a specialist prior to said step of analysing.

11. A method of investigating changes in the eye according to claim 9, wherein the mask is administered to the patient in a clinic.

12. A method of investigating changes in the eye as according to claim 9, wherein the mask is administered to the patient remote from a clinic.

13. A method of investigating changes in the eye according to claim 12, further comprising the step of transmitting the compressed digital images back to the clinic.

14. A method of investigating changes in the eye according to claim 13, wherein the compressed digital images are transmitted to the clinic via the internet.

15. A method of investigating changes in the eye according to claim 12, wherein the mask is returned to the clinic for analysis of the recorded images.

16. A method of investigating changes in the eye according to claim 12, wherein a storage medium holding the stored images is removed from the mask and transported to the clinic for analysis of the images.

17. A method of investigating changes in the eye according to claim 9, wherein the analysis of the images is aimed at diagnosing vestibular and neurological disorders.

18. A method of investigating changes in the eye according to claim 17, wherein the analysis concentrates on recorded eye movements during an attack of dizziness.

19. A method of investigating changes in the eye according to claim 18, wherein the analysis involves the identification of nystagmus during the time of a vestibular attack, as well as the intensity and direction of the nystagmus.

20. (canceled)

21. (canceled)

22. (canceled)
